(19) United States
(12) Patent Application Publication
(10) Pub. No.: US 2013/0060119 A1
(43) Pub. Date: Mar. 7, 2013

(54) DEVICE AND METHOD FOR DETERMINING FETAL MOVEMENT

(71) Applicants: Andrew Weeks, Liverpool (GB); Joan Porter, Liverpool (GB)

(72) Inventors: Andrew Weeks, Liverpool (GB); Joan Porter, Liverpool (GB)

(21) Appl. No.: 13/696,673

(22) PCT Filed: May 5, 2011
(86) PCT No.: PCT/GB2011/050879

§ 371 Date: Nov. 7, 2012

(30) Foreign Application Priority Data
May 7, 2010 (GB) 1007606.5

(51) Int. Cl. A61B 5/11

(52) U.S. Cl. 600/376; 600/588

(57) ABSTRACT

A device (100) for monitoring movement of a baby during labour is provided, comprising a movement sensing unit (210) for determining movement of a fetal scalp electrode relative to the device, and an output unit (220) arranged to output movement information indicative of the movement of the fetal scalp electrode. The movement sensing unit may comprise a rotatable member such as a wheel (410) for contacting the fetal scalp electrode (FSE) and rotating in response to linear movement thereof. In some embodiments, the device includes an optical movement sensing device for optically sensing linear movement of the FSE and outputting an electrical signal in response thereto. Preferably, the output unit (220) is arranged to wirelessly output the movement information.

### BACKGROUND

During labour or childbirth it is common for a woman to be given an epidural. An epidural reduces the woman's sensation below the waist. In the UK epidurals are given to around 20% of women during labour. The reduction in sensation is known to cause problems for women, particularly during the second stage of labour. Due to the lack of sensation, some women find it difficult to push the baby out through the birth canal. This results in approximately 20% of women requiring an operative vaginal delivery i.e. using ventouse or forceps, which carries an associated risk for the baby.

Previous systems aimed at monitoring the progress of labour included a plurality of positions sensors for attachment to the mother and baby, the locations of which in three-dimensional space are wirelessly monitored by a computer. However, such systems are complicated and consequently expensive. These systems may also not provide accurate measurements when the mother is moving.

It is an aim of embodiments of the invention to address one or more problems of the prior art. In particular, it is an aim of embodiments of the invention to provide information on the position of a baby during labour, particularly although not necessarily, to a woman giving birth.

### SUMMARY OF THE INVENTION

According to a first aspect of the invention, there is provided a device for monitoring movement of a baby during labour, comprising a movement sensing unit for determining movement of a fetal scalp electrode relative to the device, and an output unit arranged to output movement information indicative of the movement of the fetal scalp electrode. Advantageously, by determining the movement of the baby with reference to the fetal scalp electrode, more accurate measurements may be made even when the mother moves, as is expected during labour. Furthermore, since fetal scalp electrodes are frequently used during an epidural, minimal additional apparatus is required. In other words, movement of the baby is determined with by sensing movement of an existing fetal scalp electrode.

The movement sensing unit may comprise a rotatable member such as a wheel for contacting the fetal scalp electrode (FSE) and rotating in response to linear movement thereof; and a means for generating an electrical signal indicative of rotation of the wheel. The wheel may be rotatably coupled to a rotary encoder and the means for generating the electrical signal may include a light emitter and a photodetector arranged to sense rotation of the rotary encoder.

The wheel rotatable member may have a surface profile corresponding to a surface profile of the FSE. The surface profile may include a plurality of undulations arranged to generally match those present on the FSE. Advantageously, this assists in preventing slippage between the rotatable member and the FSE. In some cases the fetal scalp electrode may be formed by a wound coil and the rotatable member has a surface profile including undulations having a pitch substantially the same as the pitch of coil windings.

In some embodiments, the device includes an optical movement sensing device for optically sensing linear movement of the FSE and outputting an electrical signal in response thereto. The optical sensing may be responsive to surface features of the FSE, such as the surface profile thereof or surface markings of the elongate member.

Preferably, the output unit is arranged to wirelessly output the movement information. The output unit may wirelessly output the movement information utilising a relatively short-range wireless communication protocol. The wireless communication protocol may be Bluetooth, ZigBee or the like. The movement information may be transmitted for reception by a portable or hand-held device. The hand-held device may be for possession by the woman giving birth to provide at least nearly instantaneous feedback of the baby's movement.

The movement may indicate a distance of movement of the FSE relative to the device. In other words, the movement information may indicate the distance of movement of the FSE past the location of the device. The device may comprise attachment means for attaching to a woman's body, such that the device is held relatively stationary during use.

According to a second aspect of the invention, there is provided a method of monitoring movement of a baby during labour, comprising arranging fetal scalp electrode within a birth canal to contact the baby, determining movement of the fetal scalp electrode relative to a device within the birth canal, and outputting an indication of the movement. The indication may be output from the device.

The method may comprise attaching the fetal scalp electrode (FSE) to the baby. The FSE may then be arranged within an aperture of the device. The FSE may be arranged within the aperture by introducing the FSE to a slot within the device.

The method may comprise a step of arranging the device in a fixed position with respect to the birth canal. The fixed position may be proximal to an entrance to the birth canal.

The method may comprise a step of making an initial measurement of the location of the baby within the birth canal.

The indication of the movement of the elongate member may be wirelessly output by the device.

The step of determining the movement of the FSE may be performed with respect to one or more surface features of the FSE.

The method may comprise displaying the indication of the movement on a device viewable by a woman giving birth.

### BRIEF DESCRIPTION OF THE DRAWINGS

Embodiments of the invention will now be described, by way of example only, with reference to the accompanying figures, in which:

FIG. 1 is an illustration of an embodiment of the invention;

FIG. 2 is a schematic illustration of an embodiment of the invention;

FIG. 3 is an illustration of an embodiment of the invention arranged in relation to an elongate member;

FIG. 4 is an illustration of the elongate member and a portion of an embodiment of the invention;

FIG. 5 is an illustration of an embodiment of the invention arranged in use; and

FIG. 6 is an embodiment of a device displaying movement information.

### DETAILED DESCRIPTION OF EMBODIMENTS OF THE INVENTION

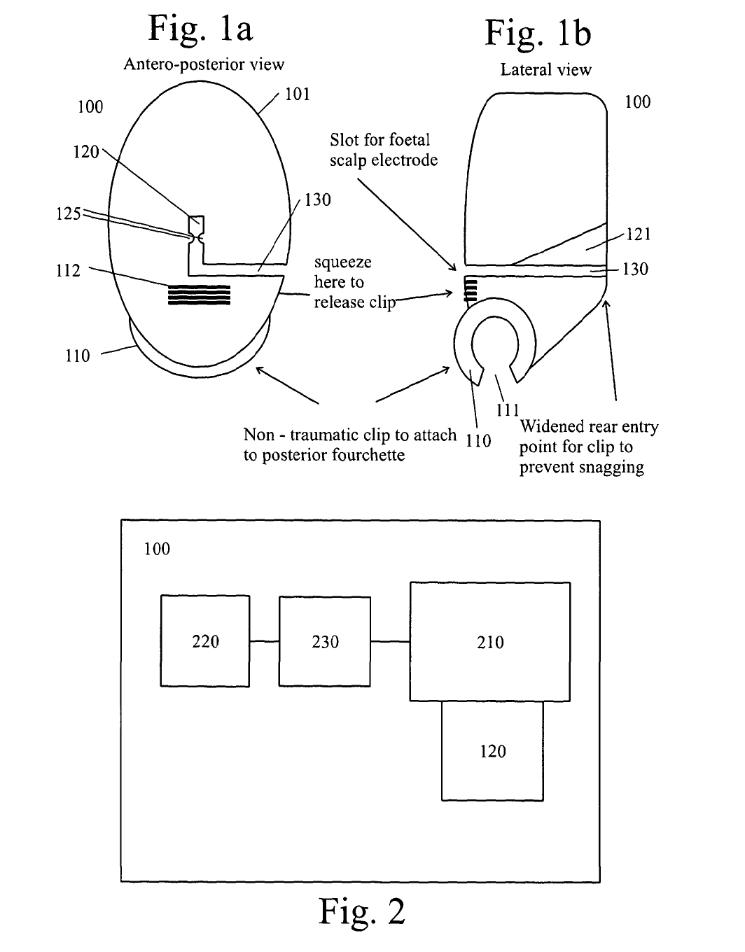

Referring to FIG. 1, a device 100 according to an embodiment of the invention is shown. FIG. 1(a) shows the device 100 in a front view, whereas FIG. 1(b) shows a lateral view of the device 100.

The device 100 comprises an exterior casing 101, typically made from moulded plastic, although other construction materials may be envisaged. Ideally the casing 101 is generally smooth to allow comfortable location of the device 100 in a woman's birth canal, as will be explained.

A clip 110 is arranged in a lower portion of the device 100 for attaching the device 100 to the woman's body during labour. Typically the clip 110 is shaped to allow attachment of the device to the woman's posterior fourchette, although other points of attachment in the woman's vaginal area may be envisaged. The clip 110 is substantially C-shaped when viewed laterally and has an opening 111 at a lower end thereof, distal from the body 101 of the device 100. The clip 110 may be made from a resiliently deformable plastic, or the like, to enable attachment and subsequent release from the fourchette. In order to assist attaching and subsequently releasing the device 100 from the woman's body, an area 112 of the casing 101 above the clip 110 may also be resiliently deformable, such that when inwardly deformed by pressure exerted by a user's finger, the opening 111 of the clip 110 widens to reduce a gripping force exerted by the clip 110.

As will be explained, the device 100 is arranged to monitor movement of an elongate member. The casing 101 of the device 100 includes an aperture 120 through which the elongate member extends during use of the device 100. The aperture 120 runs through the device 100 in an anterior-posterior or front to back direction, perpendicular to an axis of the clip 110.

A slot 130 provided in the casing 101 allows insertion of the elongate member into the aperture 120. The slot 130 extends from a side of the device 100 and is L-shaped to prevent the member from being easily released from the aperture 120 during use. Dividing the slot 130 and aperture 120 are a pair of inwardly extending projections 125. The projections are arranged on opposing faces of the slot 130 walls and inwardly project into the slot 130. Opposing ends of the projections 125 are sufficiently spaced to allow the member to move there-past with the aid of a small amount of force. The projections 125 aid retention of the member in the aperture 120.

A posterior portion 121 of the aperture 120, and also in some embodiments the slot 130, has a wider mouth than the corresponding anterior portion. In use, this allows the angular position of the device 100 to change, relative to the member passing through the aperture 120.

FIG. 2 is a schematic illustration of the device 100. The device 100 includes a movement sensing unit 210, an output unit 220 and a control unit 230.

The movement sensing unit 210 is located proximal to the aperture 120 for sensing movement of the elongate member through the aperture 120, as will be explained. The movement sensing unit 210 is arranged to output a signal indicative of the movement of the member to the control unit 230.

The control unit 230 may perform a conversion process on the signal output by the movement sensing unit 210. The control unit 230 may convert a format of the signal output by the movement sensing unit 220 to another format i.e. into a format indicative of movement of the member in predetermined units, such as millimetres. The control unit 230 outputs information indicative of the movement of the member in the predetermined units to the output unit 220. The output unit 220 outputs corresponding movement information.

In some embodiments, the output unit 220 may visually output the movement information, such as on a display device (not shown) which is connected to the device 100 by a cable. In other embodiments, the output unit 220 outputs the movement information for receipt by another device. The movement information may be output over a wired or wireless interface. A suitable wireless interface is Bluetooth, which allows the movement information to be received by one or more devices having Bluetooth communication capability, such as mobile telephones, Personal Digital Assistants (PDAs), computers (including laptop or portable computers) etc.

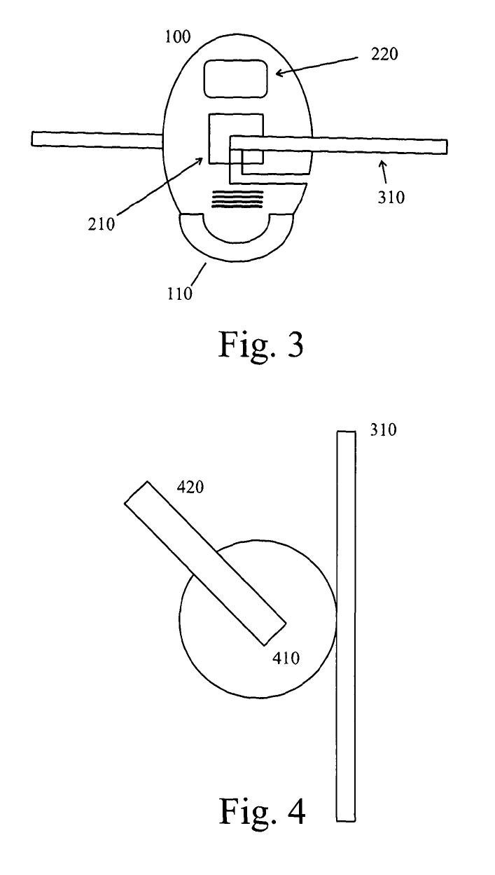

FIG. 3 is an illustration of the device 110 arranged in relation to the elongate member 310. FIG. 3 is a semi-exploded view of the device 100, in that some internal components are visible to aid explanation which would otherwise be concealed within the casing 101. The elongate member 310 is a flexible member arranged such that an end of the member 310 abuts a baby during labour i.e. as the baby travels down the birth canal. Ideally, the member 310 is resiliently flexible i.e. has at least some resilience to retain an elongate arrangement. In some embodiments, the member 310 is a fetal scalp electrode (FSE). The FSE is an electrode which is inserted into the birth canal to contact and make an electrical connection with the baby's scalp to allow measurement of the baby's heart rate during labour. Typically, the FSE if formed by a tightly wound coil of stainless steel wire having a diameter of approximately 2.13 mm, although other diameter FSEs are envisaged. Due to the formation of the FSE as a coil, the member 310 has regular indentations formed along its outer surface with a spacing of approximately 0.46 mm, although it will be realised that other spacings may be envisaged. Some embodiments of the invention utilise these indentations to assist in monitoring the movement of the FSE 310 relative to the device 100. However, in other embodiments, movement of the member 310 may be determined in other ways, such as the outer surface of the member 310 having regularly spaced optically readable markings, or electrically without the member 310 having any markings or surface features, as will be explained. Embodiments of the movement sensing unit 210 will now be described with reference as an example to the FSE.

A first embodiment of the movement sensing unit 210 determines movement of the FSE 310 by mechanically contacting the FSE 310. FIG. 4 illustrates the FSE 310 in relation to a wheel or cylinder 410 which forms part of the movement sensing unit 210 and is arranged to make contact with the FSE 310, such that linear movement of the FSE 310 is translated into rotary movement of the wheel 410.

In some embodiments, a surface of the wheel 410 has a profile corresponding to that of the FSE 310 i.e. the surface of the wheel 410 includes indentations matching those of the FSE 310 in order to ensure good mechanical contact between the member 310 and wheel 420 and to reduce slippage of the wheel 410 in relation to the FSE 310. The wheel 410 may be mounted and arranged to rotate upon one or more arms 420, such that the wheel 410 at least partly protrudes into the aperture 120. The wheel 410 may be resiliently biased against the FSE 310 by a biasing pressure applied to the one or more arms 420 by a spring or the like (not shown).

Furthermore, in some embodiments of the device 100, a cleaning device is provided to remove biological material from the surface of the FSE 310 prior to it contacting the wheel 410. The cleaning device may be a scraper arranged proximal to the surface of the FSE 310 to remove the biological material and improve adhesion between the wheel 410 and FSE 310. The scraper may, in some embodiments, be mounted upon the one or more arms 420 to also be biased against the FSE 310. The casing 101 may include a well portion into which the biological material is collected after removal from the FSE 310.

In order to convert rotation of the wheel 410 into an electrical signal output by the movement sensing device 210, a rotary encoder is coupled to the wheel 410. A rotary encoder includes a disc having openings arranged radially there-around. In some embodiments, the openings are slots evenly spaced around the disc such rotation of the disc may be detected by a photo-detector receiving light emitted from a light emitting element, such as an LED arranged on opposing sides of the disc. The photo-detector is arranged to output an electrical signal responsive to the amount of received light. In this way, rotation of the wheel 410 produces a corresponding electrical output from the photo-detector. Some embodiments of the movement sensing device 210 include a disc having two or more concentric rings of differently arranged openings and a corresponding number of pairs of light emitting devices and photo-detectors, such that a direction of movement and relative position of the FSE 310 may be determined.

In another embodiment of the device 100, the movement sensing unit 210 optically determines movement of the FSE 310. Optical determination may be performed by use of a laser device, such as a low-power ultra-violet (UV) laser. One advantage to use of such a laser is that ultra-violet light has penetrating properties that enable markings or surface features on the FSE 310 to be read even in the presence of biological material on the FSE 310. A photo-detector, such as a photo-diode, may be used to receive laser-light reflected from the FSE 310 and to output a corresponding electrical signal. As mentioned previously, in embodiments where the member 310 is a FSE, the surface of the FSE may include indentations formed between adjacent coil windings. The electrical signal output by the photo-detector responds to the locations of the indentations and interposed “peaks” formed by the windings. The control unit 230 is then arranged to determine a distance of movement of the FSE 310 according to the pitch of the windings e.g. 0.46 mm. Alternatively, the FSE 310 or other member may have optical markings thereon, such as a series of alternating light and dark regions to which the output of the photo-detector responds according to a level of reflected laser-light.

In another embodiment, the movement sensing unit 210 is arranged to receive a signal carried along the FSE 310, wherein one or more characteristics of the signal are indicative of the position or location along the FSE 310. In one embodiment, a current may be applied to the FSE 310, for example at an external end, i.e. distal from the baby. The position sensing unit 210 may determine the position of the device 100 along the FSE 310 by electrically contacting the FSE 310 to receive a voltage present at the location of the device 100 along the member 310. As the device 100 moves away from the current source along the FSE 310, the received voltage drops due to a resistance of the FSE 310. The location may be determined by the control unit 230 according to the resistance per unit length of the FSE 310.

It will be realised that embodiments of the member 310 may be envisaged in which the member 310 is not an FSE. For example, the member 310 may be a dedicated movement measurement member 310.

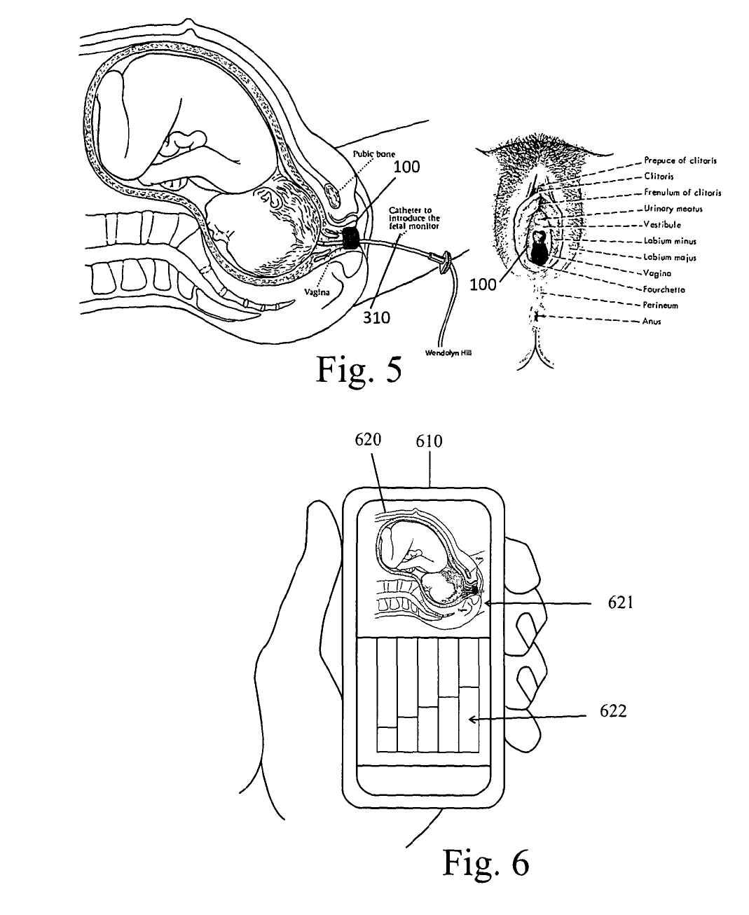

FIG. 5 illustrates an embodiment of the device 100 which is arranged in use within the birth canal. As described above, the device 100 may be releasably secured in position in the birth canal by applying pressure to the casing 101 proximal to the clip 110, such that the opening in the clip 110 widens to allow attachment of the device 100 onto the posterior fourchette, although other attachment locations may be envisaged. The member 310, such as the FSE, which is already attached to the baby's head, is then introduced to the device 100 through the slot 130 and forced past the opposed projections 125 so as to be retained in the aperture 120. As a result, the device 100 is securely held in the birth canal whilst the FSE 310 passes through the aperture 120 of the device 100 whilst abutting the baby. In some embodiments, since relative movement of the FSE 310 is only measured by the device 100, i.e. the device 100 does not determine an initial location of the baby within the birth canal, a level (distance of the baby's head from the entrance to the birth canal) may be measured prior to securement of the device 100 within the birth canal. A device suitable for measuring the level of the baby's head is disclosed in WO2007102017. Once arranged in use, the device 100 outputs an indication of the movement of the baby within the birth canal. As mentioned above, the movement may be output on a display connected to the device 100. However, in embodiments where the device 100 wirelessly outputs movement information, the movement information may be received by a mobile or portable device, such as a PDA.

FIG. 6 illustrates a PDA 610 having a display device 620, wherein the PDA 610 is arranged to receive the movement information wirelessly output by the device 100, for example via a Bluetooth connection. An application executing on the PDA 610 provides one or more visual indications on the display device 620 of the baby's movement down the birth canal and/or an indication of movement of the baby according to each pushing effort of the woman.

In one embodiment, a first region 621 of the display 620 comprises a visual indication of the baby's position within the birth canal by means of a diagram or animation including a representation of the woman's anatomy and baby. When the application is initialised, the initial level of the baby within the birth canal is entered into the PDA 610 and the diagram generated accordingly by the application. As the movement information received from the device 100 indicates that the baby is progressing down the birth canal, the diagram is updated to continuously or periodically show the new level of the baby. Thus the woman is provided with a visual indication of the progression of the baby, thereby assisting in motivating the woman during labour. A second region of the display 622 provides an indication of a distance of movement of the baby for each push of one or more pushes. In the embodiment shown in FIG. 6, the distance of movement for the last five pushes may be shown. Alternatively, the distance of movement for the last four pushes and the distance of movement during a “best” push i.e. greatest distance of movement in a single effort may be shown. Thus the woman receives instantaneous feedback on the distance of the baby's movement which serves as motivation in pushing.

It will be realised that other graphical indications may be provided on the display device 620 and that, furthermore, the usefulness of the invention is not limited to PDAs and that many other devices may be arranged to receive the movement information from the device 100 or a dedicated device may be utilised.

It will be appreciated that embodiments of the present invention provide information on a movement of a baby within the birth canal during labour. This may be particularly advantageous when the woman has had an epidural, although the usefulness of the present invention is not limited to this situation.

It will be appreciated that embodiments of the present invention can be realised in the form of hardware, software or a combination of hardware and software. Any such software may be stored in the form of volatile or non-volatile storage such as, for example, a storage device like a ROM, whether erasable or rewritable or not, or in the form of memory such as, for example, RAM, memory chips, device or integrated circuits or on an optically or magnetically readable medium such as, for example, a CD, DVD, magnetic disk or magnetic tape. It will be appreciated that the storage devices and storage media are embodiments of machine-readable storage that are suitable for storing a program or programs that, when executed, implement embodiments of the present invention. Accordingly, embodiments provide a program comprising code for implementing a system or method as claimed in any preceding claim and a machine readable storage storing such a program. Still further, embodiments of the present invention may be conveyed electronically via any medium such as a communication signal carried over a wired or wireless connection and embodiments suitably encompass the same.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The claims should not be construed to cover merely the foregoing embodiments, but also any embodiments which fall within the scope of the claims.

1. A device for monitoring movement of a baby during labour, comprising:
a movement sensing unit for determining movement of a fetal scalp electrode relative to the device;
an output unit arranged to output movement information indicative of the movement of the fetal scalp electrode.

2. The device of 1, wherein the movement sensing unit comprises:
a wheel for contacting the fetal scalp electrode and rotating in response to linear movement thereof; and
means for generating an electrical signal indicative of rotation of the wheel.

3. The device of claim 2, wherein the wheel has a surface profile corresponding to a surface profile of the fetal scalp electrode.

4. The device of 1, wherein the movement sensing unit comprises:
an optical movement sensing device for optically sensing linear movement of the fetal scalp electrode and outputting an electrical signal in response thereto.

5. The device of claim 1, wherein the output unit is arranged to wirelessly output the movement information.

6. The device of claim 1, wherein the movement indicates a distance of movement of the fetal scalp electrode relative to the device.

7-11. (canceled)

12. A system comprising the device of claim 5 and a device arranged to wirelessly receive the movement information and to output a visual indication corresponding thereto.

13. The system of claim 12, wherein the visual indication includes a graphical representation of a baby and birth canal indicative of the level of the baby within the birth canal.

14. The system of claim 12, wherein the visual indication includes an indication of a distance of movement of the baby during one or more previous pushes.

15. The system of claim 12 comprising a fetal scalp electrode, wherein the movement sensing unit of the device is arranged to determine movement of the fetal scalp electrode.

16-21. (canceled)

22. A device for monitoring movement of a baby during labour, comprising:
a movement sensing unit for determining movement of an elongate member relative to the movement sensing unit; and
an output unit arranged to output movement information indicative of the movement elongate member;
wherein the movement sensing unit comprises an attachment for attaching the movement sensing unit at least partially within a birth canal of a body.

23. The device of claim 22, wherein the attachment dip for attaching the movement sensing unit at least partially within the birth canal.

24. The device of claim 22, wherein the attachment is configured to attach to a posterior fourchette.

25. A device for monitoring movement of a baby during labour, comprising:
a movement sensing unit with an aperture configured to receive an elongate member for determining movement of the elongate member relative to the movement sensing unit; and
an output unit arranged to output movement information indicative of the movement of the elongate member;
wherein the movement sensing unit comprises a slot through which the elongate member may be passed laterally to be received by the aperture.

26. The device of claim 25, wherein the slot is L-shaped.

27. The device of claim 25 further comprising an inwardly extending projection dividing the slot and the aperture.

28. The device of claim 25 further comprising a pair of inwardly extending projections dividing the slot and the aperture.

29. The device of claim 28, wherein opposing ends of the projections are sufficiently spaced to allow the elongate member to move therepast with the aid of a small amount of force.

30. The device of claim 25, the aperture comprising a posterior portion and an anterior portion, wherein the posterior portion of the aperture has a wider mouth than the anterior portion thereof.

31. The device of claim 25, the slot comprising a posterior portion and an anterior portion, wherein the posterior portion of the slot has a wider mount than the anterior portion thereof.
